(19) United States
(12) Patent Application Publication
(10) Pub. No.: US 2009/0192365 A1
(43) Pub. Date: Jul. 30, 2009

(54) METHODS FOR ANALYSES OF HEALTH IMBALANCES AND FORMULATIONS OF CORRECTIVE DIETARY SUPPLEMENTATION

(76) Inventor: Heinz Gisel, La Jolla, CA (US)

Correspondence Address: KENNETH E. HORTON;KIRTON & MCCONKLE, 60 EAST SOUTH TEMPLE, SUITE 1800, SALTLAKE CITY, UT, 84111, US

(21) Appl. No.: 12/022,752

(22) Filed: Jan. 30, 2008

(51) Int. Cl. A61B 5/00

(52) U.S. Cl. 600/301

(57) ABSTRACT

Methods for accurately analyzing dietary nutritional deficiencies and/or imbalances in subjects, including those presumed healthy are described. The methods formulate and dispense a personalized regimen(s) of dietary nutritional supplements and/or physical programs to complement the subjects' individual needs for sustainable health. Generally, the subjects' needs are analyzed by considering a combination of health and vitality factors. Based on the analysis, a personalized regimen may be computed and formulated for the subject, wherein the regimen may include a broad range of nutrients and/or physical programs. At periodic times during the supplementation, the subject's needs may be re-assessed and, if necessary, the regimen may be adjusted. In some cases, the analysis as well as the formulation and delivery of the personalized regimen is accomplished in a single session, in a single location. In some cases, a session may be accomplished in less than about 3 hours. Other embodiments are described.

### FIELD

This application relates generally to the diagnosis and treatment of nutritional imbalances. More specifically, this application relates to methods for accurately analyzing dietary nutritional deficiencies and/or imbalances in a presumed healthy subject and methods for formulating and dispensing dietary nutritional supplements to complement the subject's individual needs for sustainable health.

### BACKGROUND

Currently, many people in the world's population suffer form deficiencies and imbalances of essential nutrients, such as vitamins, minerals, enzymes, amino acids, trace elements, fatty acids, beneficial bacteria, fibers and the like. Although some suffer from extreme imbalances and deficiencies, others suffer to a lesser extent, and may even be presumed to be healthy in the absence of disease symptoms. Nevertheless, even nutrient deficiencies in these presumed healthy individuals may cause the individuals to have sub-optimal health. For example, nutrient deficiencies may increase an individual's aging process and chances of getting sick. Moreover, such deficiencies and imbalances may reduce an individual's energy, immune response, ability to think and react, life span, senses, or cause other undesirable effects.

In an effort to reduce nutrient deficiencies and imbalances, many individuals take dietary supplements, such as vitamins, minerals, and herbal supplements. However, such supplements are often made for a broad class of individuals and are not individually formulated for the specific nutritional needs of each individual.

Some conventional methods for personalizing dietary supplementation exist and may include analyzing a patient (or subject) for deficiencies of dietary supplements, including certain vitamins, minerals, or antioxidants. However, these conventional methods have several limitations. For example, the scope of the analysis in these methods is often limited to specific tests and analyses, such as blood or hair sample tests, skin impedance measurements, body fat determinations, body mass index (BMI), or personal questionnaires. Accordingly, the analyses of many conventional methods tend to focus on particular symptoms, such as obesity or a particular nutrient deficiency, while failing to analyze the root cause of the symptom in the subject. In another example of a limitation of conventional methods, many conventional nutritional analyses fail to measure and correlate all relevant parameters. Rather, the analyses tend to calculate the quantitative need for certain nutrients without considering the compatibility or bioavailability of the combination of various supplements needed. These calculations are often based on analyses that show deficiencies of certain nutrients and do not consider the fact that such deficiencies are symptoms of a cause that cannot be identified by a single analysis.

### SUMMARY

This application describes methods for accurately analyzing dietary nutritional deficiencies and/or imbalances in any subject, including those presumed to be healthy. The methods formulate and dispense a personalized regimen(s) of dietary nutritional supplements and/or physical programs to complement the subjects' individual needs for sustainable health.

The regimen(s) or regimen may be based on a number of factors specific to the subject and based on the nutrients contained in the regimen. For instance, the regimen may be based on the subject's quantitative and qualitative need for a broad range of nutrients. The regimen may also be based on the compatibility or bioavailability of the combination of various supplements that are contained in the regimen. The regimen can consider all accessible health and vitality indicators, including, but not limited to, blood analyses, tissue analyses, genetic profiling, hair sample tests, lifestyle analyses, toxicity analysis, breath to breath analyses at rest and under stress, lung and heart function at rest and under stress, temperature profiles, microcirculation and heat generation at rest and under stress, and combinations thereof. Moreover, the regimen can consider all accessible health and vitality indicators in combination and correlation so as to look beyond the mere symptoms of a subject's conditions to find the cause.

In some cases, the regimen may be determined by software that compares the analytical results of the subject with the testing results of the subject to determine the subject's health status, potential health risks, a curriculum for averting the risks, and a personalized nutritional formulation. Further, the subject's nutritional needs may be analyzed and the personalized regimen may be delivered to the subject in one session that takes place in one location. Additionally, a session may last less than about one half of a day. Indeed, in some cases, a session may last less than about three hours. Thus, the personalized regimen may be based on a subject's health profile that is determined using both objective and dynamic analyses that are based on causes, and not merely symptoms, of factors that can detract from the subject's actual and sustainable health.

### BRIEF DESCRIPTION OF THE DRAWINGS

In order that the manner in which the above recited and other features and advantages of the described methods are obtained, a more particular description of the methods for analyzing dietary deficiencies and/or imbalances in a subject and methods for formulating and dispensing personalized regimens of dietary nutritional supplements and/or physical programs will be rendered by reference to some embodiments thereof, which are illustrated in the appended drawings. Understanding that the drawings depict only some embodiments of the described methods and are not, therefore, to be considered as limiting the scope of the methods, the methods will be described and explained with additional specificity and detail through the use of the accompanying drawings in which:

FIG. 1 contains a flow chart showing some embodiments of the methods for analyzing dietary deficiencies and/or imbalances in a subject and methods for formulating and dispensing a personalized regimen of dietary nutritional supplements and/or physical programs;

FIG. 2a contains a flow chart showing other embodiments of these methods;

FIG. 2b contains a flow chart showing yet other embodiments of the data flow for these methods; and

FIG. 3 contains a flow chart showing still other embodiments of the described methods.

Together with the following description, the Figures demonstrate the features of the methods for analyzing dietary deficiencies and/or imbalances in a subject and methods for formulating and dispensing a personalized regimen of dietary nutritional supplements and/or physical programs. In the Figures, the thickness and configuration of components may be exaggerated for clarity. The same reference numerals in different Figures represent the same component.

### DETAILED DESCRIPTION

The following description supplies specific details in order to provide a thorough understanding. Nevertheless, the skilled artisan would understand that the disclosed methods for analyzing dietary deficiencies and/or imbalances in a subject and methods for formulating and dispensing a personalized regimen of dietary nutritional supplements and/or physical programs can be implemented and used without employing these specific details. For example, while the description focuses on implementing the described methods with presumed healthy humans, it can be modified for implementation with humans who are known to be not healthy. Moreover, the described methods may be modified enhance performance of a subject, such as an athlete. Additionally, the described methods may be modified for use with equine, canine and other animals.

The methods for analyzing dietary deficiencies and/or imbalances in a subject and methods for formulating and dispensing a personalized regimen(s) of dietary nutritional supplements and/or physical programs are based on the subject's individual needs. Generally, the subject's needs are first analyzed by considering a combination of health and vitality factors. Based on the analysis, a personalized regimen is computed and formulated for the subject, wherein the regimen may include a broad range of nutrients and/or physical programs. The nutrients are used by the subject according to the regimen so as to improve or restore the subject to optimal health over a period of time. At periodic times during the supplementation, the subject's needs may be re-assessed and, if necessary, the regimen may be adjusted.

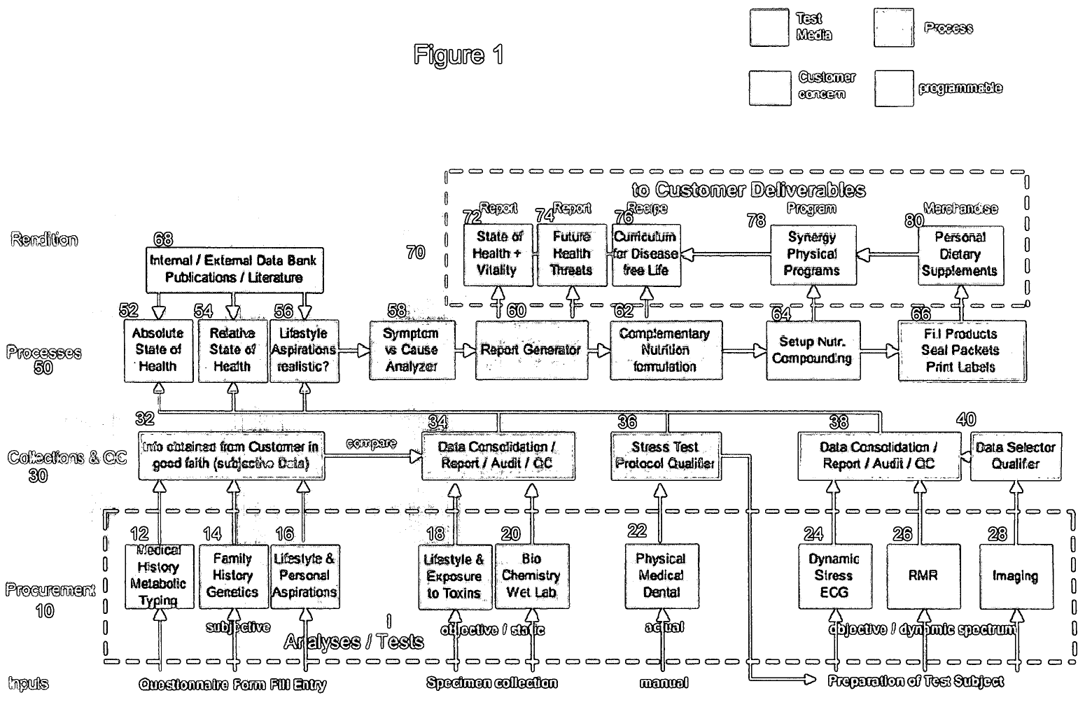

FIG. 1 illustrates some embodiments of these methods. Although any number or types of health and vitality factors can be considered when analyzing the subject's nutritional needs, row 10 in FIG. 1 shows some non-limiting examples of health and vitality factors, including the subject's medical history, metabolic typing, family history/genetics, lifestyle, personal aspirations, exposure to toxins, current health status, the subject's exposure to free radicals and oxidants, the subject's vitality related goals, and combinations thereof.

FIG. 1 shows some embodiments where the health and vitality factors considered for determining a subject's nutritional needs may include the subject's medical history, as shown in box 12. Such information may include any data relating to the subject's past health, including, but not limited to, prior disease, prior surgery, prior medical treatment, prior diagnosis, prior medications, prior dietary supplements, congenital defects, and combinations thereof.

Box 12 also shows that the subject's metabolic typing may be considered as a heath and vitality factor. Information about the subject's metabolic typing may include any data that relates to the subject's individual metabolism. Some non-limiting examples of data that may relate to the subject's individual metabolism may include data about the subject's physical condition (e.g., aging, chest pressure, cold sores/fever blisters, coughing, dandruff, digestion, eye moisture, facial coloring, facial complexion, reaction to fatty foods, fingernail condition, gum bleeding, gooseflesh, hunger feelings, reaction to insect bites, saliva amount and texture, skin moisture, sneezing, and combinations thereof) and the subject's behavior (e.g., appetite, aloofness, climate preference, desire for sweets, desire for fatty foods, eating habits, fasting habits, meal portions, emotional expression, emotions, desire for salty foods, snacking habits, desire for sour foods, social expression, energy level, and combinations thereof).

Box 14 shows that the subject's family medical history and genetic information may be considered as a health and vitality factor when determining the subject's nutritional needs. By way of non-limiting example, such information may include data about past health, disease, life span, defects, etc. of ancestors, siblings, posterity, and other close relatives. Additionally, such information may include data about the specific genetic code of the subject (e.g., DNA markers for disease, such as cancer).

As shown in boxes 16 and 18, other examples of health or vitality factors that can be considered when determining the subject's nutritional needs may include information about the subject's lifestyle, exposure to toxins, and personal aspirations. Some non-limiting examples of data concerning the subject's lifestyle may include data about the subject's place of residence (e.g., altitude, temperature, humidity, and combinations thereof), food and dietary consumption (e.g., the types of food consumed, such as grains, red meats, fruits, vegetables, fatty foods, and combinations thereof), eating habits, stress level, occupation, tobacco products and alcohol consumption, and combinations thereof. Some non-limiting examples of data concerning personal aspirations may include information about the subject's desired health and activity level (e.g., desire to improve physical abilities, run a marathon, lose weight, gain muscle, exercise regularly, complete physical therapy, and combinations thereof). Moreover, non-limiting examples of data related to toxin exposure can include data about the subject's occupation (e.g., miner, crop duster), residence (e.g., proximity to industry), alcohol consumption, drug use, cigarette use, personal care item use, tap water use, breathing air condition, smog, contaminated foods, metals in the mouth from previous teeth restoration, and combinations thereof.

Additional health and vitality factors that may be considered when determining the subject's nutritional needs may include any information relating to the subject's current health status. Some non-limiting examples of such information can include data about the subject's age, sex, weight, height, fitness level, activity level, current health (e.g., current disease, syndromes, symptoms), race, ethnicity, mental health, emotional health, oxygen consumption, carbon dioxide production, cholesterol level, blood sugar level, blood pressure, cardiovascular health, respiratory health, skin health, hormone balance, digestive process, digestive tract health, kidney and liver function, urinary tract health, nervous system function, lymphatic system health, oral health (i.e., the health of teeth and gums), skeletal health, tissue health, resting metabolic rate (“RMR”), body mass index, waist girth, aerobic capacity, hematology, endocrinology, glucose levels, insulin levels, HDL and LDL levels, triglyceride levels, hormone function, lymphatic function, glandular function (e.g., thyroid and adrenal), homocysteine levels, CRP, sensory system condition (i.e., hearing and vision), nutritional (or dietary) deficiencies, malnutrition, and combinations thereof Other health and vitality factors that may be considered are depicted in FIG. 1.

The subject's health and vitality information may be procured in any known manner depending on the health and vitality factor being procured. For example, FIG. 1 shows that information about the subject's medical history and metabolic typing 12, family history/genetics 14, and life style/aspirations 16 may be procured through questionnaire. FIG. 1 also shows that some information relating to the subject's lifestyle/exposure to toxins 18 and current health may be procured through specimen collection. Some non-limiting examples of specimens that may be collected may include blood, urine, stool, tissue, cells, saliva, hair, fingernails, skin, and combinations thereof. Such specimens may be used or tested in any desired manner, including to for pH determination, blood analyses, tissue analysis, genetic profiling, hair sample tests, lifestyle and exposure to toxic elements analyses, saliva testing, urine analysis, body composition and body fat analysis, metabolic typing, and combinations thereof.

Box 22 shows that additional health and vitality information relating to the subject's health status may be obtained manually (e.g., by a doctor or nurse). By way of non-limiting example, such manual procurement may include physical examination of the subject's skin, mouth, hearing, vision, tactile and nervous system, speech, organs, mental health, lung function, heart function, senses, nervous system function, and combinations thereof.

In addition to the previously discussed methods for procuring health and vitality information from the subject, such information may be obtained through any other known method. For example, as shown at box 24 in FIG. 1, a resting and stressed heart function and circulatory analysis may be obtained through one or more electrocardiograms (“ECG”). Similarly, as shown at box 26, the subject's resting metabolic rate may be determined through any known method. Further, exhaled breath may be analyzed (“EBA”) for volatile organic compounds (“VOC”) or traces of disease indicators. Additionally, a functional analysis of the lungs at rest and under stress may be performed by spirometry; mitochondrial energy production capacity at rest and under stress may be determined by a breath to breath analysis; skin, bone, joint, and internal health may be determined by appropriate imaging (e.g., forms of imaging that do not involve ionizing radiation, such as thermal imaging, thermography, ultrasound, endoscopy, etc.), as shown at box 28; or a temperature profile, indirect calorimetry, oximetry, microcirculation analysis, and heat generation at rest and under stress may be performed, as known in the art.

In some embodiments, some health and vitality factors may be procured through the measurement of the subject's oxygen consumption and carbon dioxide production over a certain time period, both at rest and under stress (e.g., at a forced elevated heart rate). Such measurements may serve as a primary indicator of cellular health, including but not limited to the subject's resting and maximal mitochondrial efficiency, resting and maximal fat metabolism, caloric intake, lung function, heart function, overall strength, optimal exercise level, and combinations thereof.

Row 30 of FIG. 1 shows that, in some cases, the procured health and vitality information may be collected and tested for quality control purposes. For example, the information obtained from the subject (or customer) in good faith, as shown in box 32, may be compared for conformity and accuracy with the laboratory results obtained and consolidated from specimen collection and testing, as shown at box 34. Moreover, box 36 shows that a stress test protocol qualifier may be used to determine whether additional tests should be performed on the subject or whether the data collected through manual procurement is ready for processing. Boxes 38 and 40 show that objective/dynamic information that was obtained (e.g., through ECG 24, RMR 26, and/or imaging 28) may be consolidated, reported, audited, and checked for quality control. In another example (not shown), the results of the oxygen consumption and carbon dioxide production analysis may be compared for accuracy with findings in traces of exhaled breath (“EBA”), or in the blood, urine, and/or other lab tests (e.g., physiological tests, physical examination, lung function by spirometry, heart function by ECG, circulation by infrared [“IR”] or ultrasound imaging), and with personal data obtained from the subject.

As shown in row 50 of FIG. 1, some or all the results from the entire battery of tests can be processed to determine the health status of the subject. The processing may be done in any known manner. For example, the processing may be done by a software program. Such a software program may be any computer program product that contains computer readable medium with program code that can be utilized to carry out these methods. The computer program code can comprise any known executable code directed to perform the methods described herein.

In some embodiments, the software program compares the dynamic analyses (such as breath-to-breath at rest and under stress, the exhaled breath compounds, and/or the imaging data) with those analyses assessed from specimens (such as blood, urine, saliva, tissue, sweat, and combinations thereof) and with the data obtained from the subject's enrollment forms for consistency and for pinpointing causes. By way of non-limiting example, if traces of acetone are found in a subject's exhaled breath, the software may search for confirmation of diabetes in the lab results of insulin, glucose and other parameters in blood and/or urine. It may also search the enrollment forms for the subject's disclosure and awareness of this information. In another example, if the breath-to-breath analysis cites a subject's low CO₂ production, the cause of this symptom may be searched for by comparing lab data (such as Thyroid function, liver profile, kidney profile, adrenal function, cortisol, and combinations thereof) and/or by comparing heart function assessed by ECG, breath-to-breath stress test, and/or by exhaled breath analysis of ethane, the marker of oxidative stress, and/or by comparing the subject's enrollment form, and/or notes and comments by physician or nurse. Further, the software may seek to confirm lab data that indicates reduced liver function with exhaled breath analyses for carbonyl sulfide (COS), and/or it may seek to quantify the damage by scanning the stress test charts or another similar source. The software may find such information from a suitable source, including an internal or external data store that contains publications, literature, or results from other tested subjects, as shown in box 68. In this manner, the software may be able to determine deviations in the subject, and the causes of such deviations, from established normative standards in the data store.

In one example of how the results are processed, the dynamically assessed physical data of the subject can be compared with laboratory analyzed results of other subjects. For instance, the subject's testing results related to glucose, insulin, lipid profile (i.e., HDL, LDL cholesterol, and triglyceride levels), liver function, kidney function, mineral levels, hormone function, lymphatic function, glandular function (e.g., thyroid and adrenal), hematology, pH, homocysteine, and/or CRP may be compared with the results considered to be normal by the medical establishment. This comparison and processing may show deviations from the considered normal values.

This comparison process can generate numerous results, including a health status (both absolute 52 and relative 54) of the subject. Additionally, FIG. 1 shows that the results may indicate whether the subject's desired lifestyle and aspirations are realistic in light of the health status and physiology, as shown in box 56.

In some embodiments, the result(s) processing (e.g., via the software) may use the health and vitality factors to look beyond the mere symptoms of a subject's conditions so as to focus on the causes of manifest symptoms as shown in box 58. For example, instead of focusing on symptoms, such as obesity, diabetes, metabolic syndrome, lack of energy and aging, high blood pressure, cholesterol, and/or gum disease, the cause of the symptoms may be determined. In this manner, the cause of the symptom can be treated, as opposed to just the symptom itself.

In some embodiments, the software may include a report generator (as shown in box 60) that produces a report concerning the subject's state of health and vitality, as shown in box 72. The report generator may also produce a report concerning future health threats (as shown in 74) to the subject that may include an analysis of the subject's vulnerability to general or specific disease or ailment.

After some or all of the subject's health and vitality factors have been considered, whether or not they relate to dietary supplements, a personalized regimen of dietary supplementation and/or physical programs may be customized for the subject. Indeed, in some cases, the regimen may account for substantially all the analyzed health and vitality factors.

In some instances, as shown at box 62, the comparison of the subject's results can be used to translate the normative divergence into a formulation of nutritional supplements. This nutritional formula may be specifically created for the subject so as to substantially match the subject's nutritional needs and health risk profile. Accordingly, the nutritional formula may improve or restore the health of the subject.

In some cases, the nutritional formula is based on the causes, and not the symptoms, of the subject's determined nutritional deficiencies or imbalances. For example, rather than just considering the symptom of a lack of energy and then concluding that an energy drink (such as REDBULL®) can be part of the nutritional formula, the nutritional formula can be based on the factors that cause or contribute to the lack of energy (e.g., an inability to sleep well) and can then recommend a nutritional formula that will treat the cause of the lack of energy (e.g., the lack of sleep), rather than the manifest symptom of the lack of energy.

Indeed, because supplementation of a single nutrient based on an analyzed deficiency may lead to an aggravated imbalance of other nutrients in the subject, it may be beneficial to analyze the results from the analyses of more than one, if not all, of the subject's health and vitality factors in order to pinpoint the cause of the analyzed nutrient deficiency. For example, it may be inappropriate to treat the subject's high blood pressure with a blood pressure reducing diet without first analyzing the cause of the subject's high blood pressure. In this example, a review of the subject's analyzed health and vitality factors may reveal the cause of the high blood pressure is reduced kidney and/or digestive organ function, as opposed to the subject's diet. Accordingly, the nutritional formula may be created to treat the reduced kidney function.

Another example of the importance of treating the cause instead of the symptom may be seen where a blood or hair sample test shows a subject has a calcium deficiency. Under a symptoms based treatment, this would lead to supplementing calcium, which could make the situation worse. However, under the methods described herein, the results from the analysis of multiple health and vitality factors may show the cause of the calcium deficiency to be impaired digestion. Accordingly, the subject may need to be supplemented with HCL and digestive enzymes to allow the absorption of calcium from the food consumed.

The amounts and types of one or more nutritional supplements (“NS”) included in the nutritional formula may depend on numerous factors. Indeed, the types and amounts of the NS included in the nutritional formula may be based on any factor that aids the nutritional formula to improve the health of the subject.

For example, the amount of the NS may depend on factors such as the subject's weight, height, age, metabolism, percent body fat, specific lifestyle issues (e.g. smoking, stress and environment), specific health and vitality factors, overall health status, the extent of the subject's nutritional deficiency or imbalance, and combinations thereof. Additionally, the amount of any given NS may depend on the subject's individual diet and the other types of NS that are being considered, as discussed herein.

The types of NS that can be included in the nutritional formula are virtually limitless. Some non-limiting examples of suitable types of the NS may include any suitable form of vitamin, co-factor, mineral, trace element, fatty acid, enzyme, fiber, protein, amino acid, hormone, antioxidant, herbal supplement, beneficial bacteria, fiber, and combinations thereof.

In some embodiments, the nutritional formula in the regimen may not only be based on the number and types of the NS, but may also be based on the compatibility or bioavailability of the combination of various supplements that are contained in the nutritional formula. For instance, because some types of the NS may be incompatible or mutually exclusive for use in the body, the nutritional formula may be formulated to avoid such combinations. Similarly, because some of the types of the NS cannot be absorbed when delivered in isolated forms, the nutritional formula may include co-factors, one or more complimentary types of the NS to increase absorption, as shown at box 64. In some cases, the nutritional formula may include any suitable absorption accelerant or otherwise complimentary NS. For example, when the nutritional formula contains calcium, complimentary NS, such as magnesium and vitamin D, may be added to the nutritional formula to increase the absorption of calcium.

In some embodiments, the regimen is also based on how dietary supplements work synergistically with environmental influences. Some non-limiting examples of environmental influences may include increased or decreased exposure to sunlight, inhalation, exhalation, physical activity, transdermal absorption of substances, and combinations thereof. These environmental influences may serve many purposes, including acting as absorption accelerants or causing the body to produce or retain the desired nutrients.

The nutritional formula can be used in combination with any complimentary physical program. For example, FIG. 1 illustrates embodiments where the regimen includes personal nutritional formula (or personal dietary supplements) 80 as well as a synergistic physical program 78 that incorporates one or more environmental influences (e.g., regular exercise, more or less exposure to sunlight, etc.) that work synergistically with the supplements. By way of non-limiting example, the supplementation of L-Camitine may be beneficial for increasing exercise performance, while it has no beneficial effect on sedentary subjects. Or, as another example, anaerobic exercise is associated with joint pain, which can be eliminated by supplementation with essential fatty acids. Furthermore it is well known that sustainable, healthy weight reduction is necessarily associated with exercise and reduction in caloric intake, therefore a rebalance of nutrition may be essential to address the cause that led to excessive weight in the first place.

In some embodiments, the personal regimen (i.e., the nutritional formula) may also include components for the elimination of toxins (e.g. heavy metals) from the subject. As known in the art, toxins can be detrimental to the subject's health. Accordingly, the nutritional formula can be specifically formulated to contain any suitable component that will interact/react with toxins in the subject's body so as to render the toxins inert, reduce their detrimental impact, and/or to remove them from the body. By way of non-limiting example, the nutritional formula may include potassium iodide for a subject who has been exposed to radioactive materials.

The nutritional formula can be delivered to the subject (such as a patient) through any mechanism known in the art, including through oral delivery or non-oral delivery methods. Some non-limiting examples of suitable forms of the nutritional formula that may be delivered orally may include pills, capsules, granules, powders, enriched food products, drinks, elixirs, drops, and combinations thereof. Some non-limiting examples of suitable forms of the nutritional formula that may be delivered non-orally include forms that may be delivered intravenously (“IV”), transdermally, transocularly, topically, and combinations thereof. Additionally, the nutritional formula may be administered to the subject in a single dose, or in multiple doses over a period of time.

As shown in box 66 in FIG. 1, once formulated, the nutritional formula may be produced, packaged, sealed, and labeled. Then, as shown at box 80, the dietary supplements produced specifically for the subject may be delivered to the subject. While the personalized dietary supplements may be delivered to the subject from any appropriate location and in any suitable time frame, in some embodiments, the dietary supplements are delivered to the subject at the facility where the subject's health and vitality factors were analyzed and during the same visit as the analyses.

As shown at box 76, the nutritional formula from boxes 62 and 64 and/or a synergistic physical program, as shown in box 78, may be incorporated into a personalized regimen (or curriculum) for the subject. When followed, this personalized regimen may help to modify the health of the subject and allow the subject to avoid future health threats, such as illnesses and disease. Thus, in embodiments where the personalized regimen is followed, it may serve to increase and/or restore the subject's health or to prevent and/or cure certain illnesses.

In some embodiments, after the subject has followed the regimen for a desired period of time, the subject may be tested or retested to determine the efficacy of the regimen and/or to determine if and/or how the regimen should adjusted. In such embodiments, the efficacy of the regimen may be measured in any suitable way. However, because the measurement results regarding the presence of nutrients in the subject may change from day to day and depend on recently ingested foods, such tests may not properly reflect the effectiveness of the regimen. Instead, the effectiveness of the nutritional formula and/or physical program can be objectively and reliably assessed by measuring the increase of mitochondrial energy production in correlation with some or all other test results.

In some embodiments, the subject's health and vitality factors are collected and analyzed by a battery of analytical tests (including those described above) and the personalized regimen is produced and delivered to the subject in a single location and in a single session. In some instances, the regimen can be personalized in a short time frame, i.e., within minutes after the battery of tests have been run. Thus, such a session (beginning with the collection of health and vitality information and ending with the delivery of the personalized regimen) may last less than half a day. Indeed, in some instances, the session lasts less than about 5 hours. In other instances, however, the session lasts less than about 3 hours and occurs in a single location.

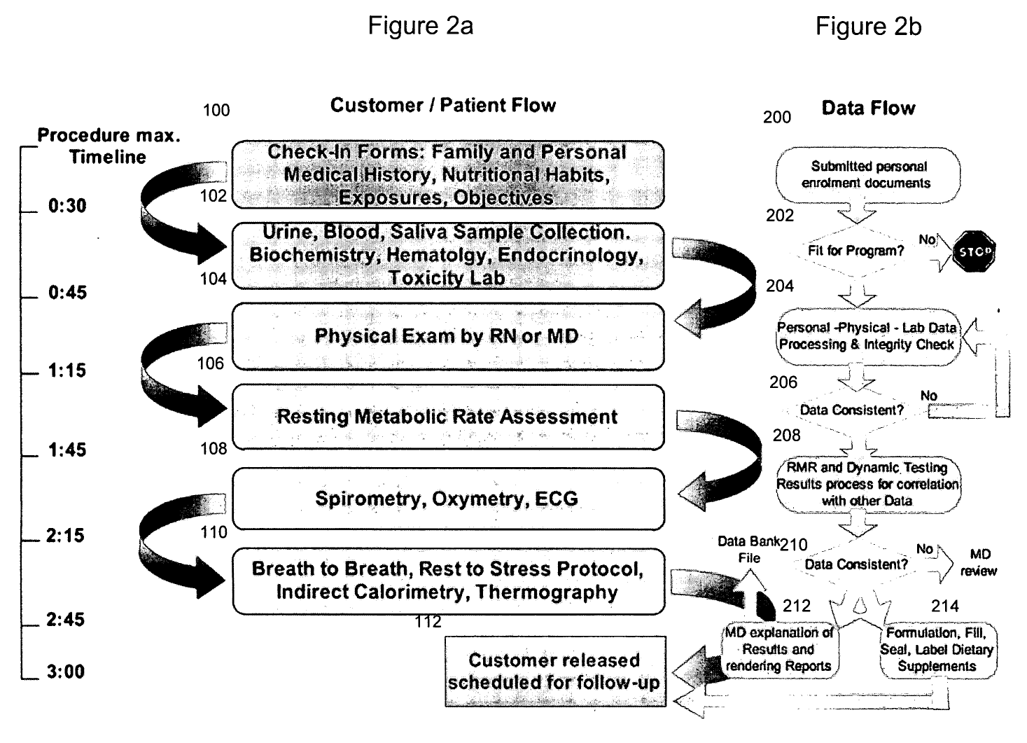

By way of non-limiting example, FIG. 2a shows some embodiments of a single session in which the analytical tests are performed and the nutritional formula is delivered to the subject in about 3 hours in a single location. Although the various elements may be performed in more or less time, box 100 of FIG. 2 shows that the subject may fill out check-in forms relating to family and personal medical history, nutritional habits, exposures, and objectives in about 30 minutes. Box 102 shows that urine, blood, and saliva collections, along with biochemistry, hematology, endocrinology, and toxicity labs may all be performed within about 15 minutes. Next at box 104, a physical examination by a nurse or doctor may be performed on the subject less than about 30 minutes. Then at box 106, a resting metabolic rate assessment may be performed on the subject in less than about 30 minutes. Following that assessment, spirometry, oximetry, and ECG analyses may be performed on the subject in less than about 30 minutes. Next, at box 110, breath to breath, rest to stress protocol, indirect calorimetry, exhaled breath, and thermography analyses may be performed in less than about 30 minutes. Finally, at box 112, the client may be released and a follow-up visit may be scheduled in less than about 15 minutes.

FIG. 2b exemplifies the data flow during a 3 hour session in these embodiments. Specifically, at box 200, FIG. 2b shows the personal enrollment documents may be submitted. From the personal enrollment information, at box 202, a determination may be made as to whether the subject is fit for the program. If the subject is determined not to be fit for the program, the session may be ended. However, if the subject is fit for the program, the subject's personal, physical, and laboratory data may be collected and compared to ensure the integrity of the system. If at box 206 the data is determined not to be consistent, the processes in box 204 may be repeated. Conversely, if the data is determined to be consistent, then at box 208, the subject's resting metabolic rate and dynamic testing results may be processed and correlated with the other data that has been collected. If that data is not determined to be consistent at box 210, then it may be reviewed (e.g., by a doctor) to determine the cause of the inconsistency. However, if the data is consistent then reports may be generated and the results and reports may be delivered and explained to the subject, as shown at box 212. The results may then be sent to a data bank for future comparisons for subsequent subjects. Additionally, the nutritional formulation may be formulated, filled, packaged, and labeled, as shown at box 212, for distribution to the subject before release.

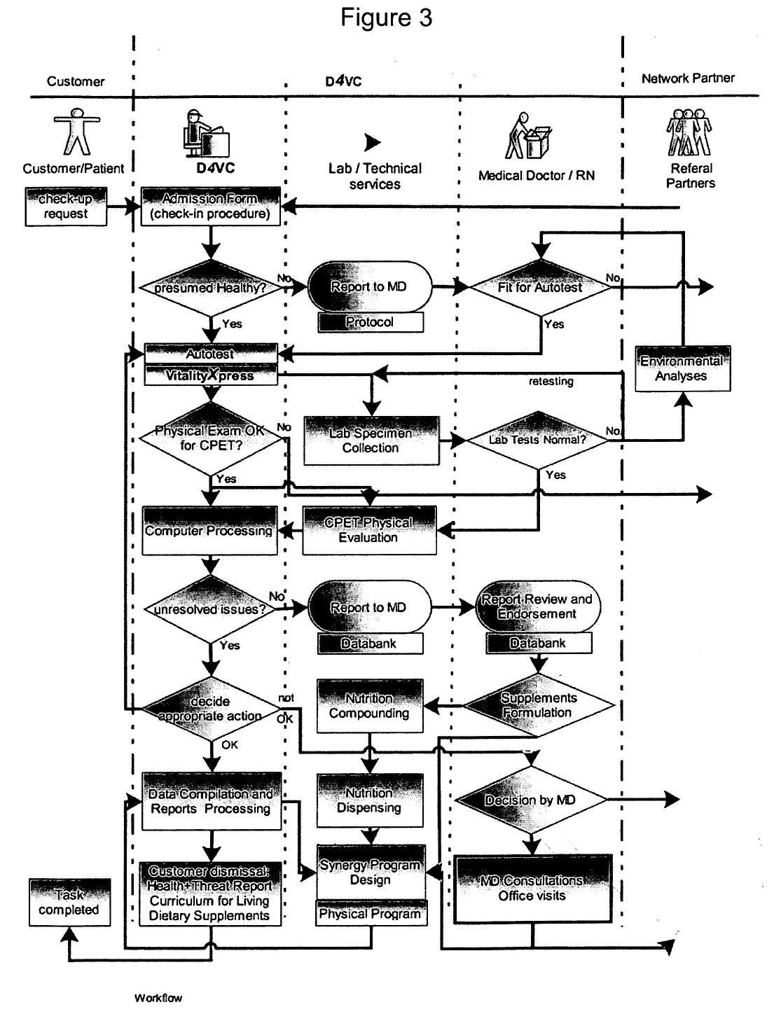

Although the described methods may be performed in any manner that achieves the intended purposes, FIG. 3 shows some additional embodiments of a process that can be followed to determine the subject's nutritional needs and to fill those needs. Specifically, FIG. 3 shows some examples of processes and tasks that may be followed during a session and delegates those tasks so they can be performed by various personnel. For instance, FIG. 3 illustrates the tasks that of the receptionist (“D4VC”), lab/technical services personnel, medical staff (e.g., doctor or nurse), and referral partners during a session.

The methods described above may offer several benefits and advantages over conventional methods for personalizing dietary supplementation. For example, as mentioned, the described methods may focus on more than one health or vitality factor and, thereby, provide the subject with a more comprehensive treatment. Furthermore, the methods may focus on determining and treating the underlying cause of a symptom, instead of the symptom itself. Additionally, the methods specifically address the individual needs of the individual subject, as opposed to a broad class of people. Moreover, the methods may consider the compatibility and bioavailability of the various NS in the nutritional formulation and the importance of environmental influences in their effectiveness. And the analysis of the subject's nutritional needs and the formulation and delivery of a personalized regimen (i.e., a personalized nutritional formula and/or physical program) may be accomplished in a short period of time (e.g., in less than about 3 hours) and in a single location.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, examples are meant to be illustrative only and should not be construed to be limiting in any manner.

1. A method for modifying the health of a subject, comprising:
analyzing the nutritional needs of a subject by considering a combination of various health and vitality factors;
preparing a personalized regimen for the subject, the regimen including a customized nutritional formula; and
administering the regimen to the subject over a period of time.

2. The method of claim 1, wherein the personalized regimen further comprises a synergistic physical program.

3. The method of claim 1, further comprising re-analyzing the patient's needs and, if necessary, adjusting the regimen.

4. The method of claim 1, wherein the nutritional formula comprises multiple nutritional supplements that are compatible with each other or that increase the bioavailability of the nutritional supplements.

5. The method of claim 1, wherein the personalized regimen treats the cause, instead of the symptom, of a health imbalance of the subject.

6. The method of claim 1, wherein analyzing the nutritional needs of the subject and preparing a personalized regimen for the subject is accomplished in a single location in a single session.

7. The method of claim 6, wherein the session last less than 3 hours.

8. The method of claim 1, wherein the analysis comprises a combination of objective and dynamic testing including a manual physical examination, an analysis of body fluids, and a time and stress sensitive profile.

9. The method of claim 8, wherein results from the analysis are processed to determine the cause for deviation in the subject from established normative standards.

10. The method of claim 1, wherein the personalized regimen compensates for assessed imbalances and is adapted to avert future health risks.

11. A method for modifying the health of a subject, comprising:
analyzing the nutritional needs of a subject by considering a combination of various health and vitality factors comprising a manual physical examination, an analysis of body fluids, and a time and stress sensitive profile;
determining the cause for deviation in the subject from established normative standards;
preparing a personalized regimen for the subject, the regimen including a customized nutritional formula and a synergistic physical program; and
administering the regimen to the subject over a period of time.

12. The method of claim 11, wherein the nutritional formula comprises multiple nutritional supplements that are compatible with each other or that increase the bioavailability of the nutritional supplements.

13. The method of claim 11, wherein the personalized regimen treats the cause, instead of the symptom, of a health imbalance of the subject.

14. The method of claim 11, wherein analyzing the nutritional needs of the subject and preparing a personalized regimen for the subject is accomplished in a single location in less than 3 hours.

15. A computer-implemented method for restoring or improving the health of a subject, the method comprising:
a computer readable medium for providing computer program code means utilized to implement the method, the computer program code comprising executable code for comparing analytical laboratory results from a subject with testing results to determine the nutritional needs of the subject and then computing a personalized regimen for the subject, the regimen including a customized nutritional formula.

16. The method of claim 15, wherein the personalized regimen further comprises a physical program that works synergistically with the nutritional formula.

17. The method of claim 15, wherein the computer program code further comprises executable code for providing the subject with a report that addresses a state of the subject's of health and vitality, future health threats to the subject, and a curriculum for the subject to live a disease free life.

18. The method of claim 15, wherein the nutritional formula comprises multiple nutritional supplements that are compatible with each other or increase bioavailability of the nutritional supplements.

19. The method of claim 15, wherein the comparison determines deviations in the subject from established normative standards.

20. A computer-implemented system for restoring or improving the health of a subject, the system comprising:
a data store for storing information about health and vitality factors; and
a computing device in communication with said data store that is operative to:
analyze the nutritional needs of a subject by considering a combination of various health and vitality factors; and
prepare a personalized regimen for the subject, the regimen including a customized nutritional formula and a physical program that works synergistically with the nutritional supplement formulation.

21. A personalized treatment regimen for modifying the health of a subject, comprising:
a nutritional supplement formulation containing multiple nutritional supplements, the formulation being customized for a subject based on that subject's health and vitality factors and the formulation based on the subject's nutritional needs and health risk factors; and
a physical program that works synergistically with the nutritional supplement formulation.

22. The treatment regimen of claim 21, wherein the nutritional formula comprises multiple nutritional supplements that are compatible with each other or that increase the bioavailability of the nutritional supplements.

23. The treatment regimen of claim 21, wherein the personalized regimen treats the cause, instead of the symptom, of a health imbalance of the subject.
